(19) United States
(12) Patent Application Publication
(10) Pub. No.: US 2015/0076973 A1
(43) Pub. Date: Mar. 19, 2015

(54) FURNITURE FOR AND METHOD OF DISPENSING DIAPERS

(71) Applicant: Donald A. PIERSON, San Diego, CA (US)

(72) Inventor: Donald A. PIERSON, San Diego, CA (US)

(21) Appl. No.: 14/489,361

(22) Filed: Sep. 17, 2014

Related U.S. Application Data
(60) Provisional application No. 61/880,123, filed on Sep. 19, 2013.

(51) Int. Cl. A47B 83/04; A47F 1/06; A61F 13/84

(52) U.S. Cl. CPC A47B 83/045; A61F 13/84; A47F 1/06
USPC 312/71

(57) ABSTRACT

Disclosed herein are a piece of furniture and a method for dispensing diapers. The furniture keeps diapers neatly organized and conveniently accessible for dispensing. The furniture has an elongated interior space, a platform, a lead screw, a motor, a detector, and a processor. The platform can hold a stack of diapers and move the stack of diapers so they can be dispensed from the top of the piece of furniture. Rotating a lead screw raises and lowers the platform within the piece of furniture. A detector positioned at the top of the interior space of the piece of furniture is configured to detect when a diaper is dispensed from the interior space. A processor in communication with the detector and the motor is configured to activate the motor, thereby raising the platform, when the detector detects that a diaper has been dispensed from the piece of furniture.

### CROSS-REFERENCE TO RELATED APPLICATIONS

This application claims the benefit of U.S. Provisional Application No. 61/880,123, filed Sep. 19, 2013, the disclosure of which is herein incorporated by reference in its entirety.

### FIELD

The present disclosure pertains generally to furniture, and more specifically to a piece of furniture for and a method of storing and dispensing diapers. In one embodiment, the furniture keeps diapers neatly organized and conveniently accessible for dispensing.

### BACKGROUND

Changing diapers can be difficult process, especially when a baby is moving and crying. The person changing the diaper may want to do this task quickly as possible. The diaper often needs to be reached with one hand while the other hand is occupied with the baby.

### SUMMARY

In addition to removing a diaper from storage easily, it is desirable to keep the stock of diapers organized and conveniently accessible for future use. Therefore it is the object of the present disclosure to each diaper storage, retrieval, and tracking. A diaper stock can be conveniently located so that a clean diaper is accessible within arm's reach. The diaper storage can also be replenished and readied for the future use. With the challenges of diaper changing, the present disclosure aims to make the diaper changing process easier and quicker.

The present disclosure provides a diaper dispensing method and system.

The present disclosure further provides a piece of furniture that holds a stack of diapers on a platform. In some embodiments, the piece of furniture vertically adjusts the stack of diapers. One aspect of the adjustment feature allows a person changing a baby to have a clean diaper conveniently located, for example, within arm's reach.

The present disclosure further provides a device and method for lifting diapers or a stack of diapers. In one embodiment, the diapers or stack of diapers is lifted within the provided piece of furniture.

The present disclosure further provides for a device and method for detecting the removal of a diaper from a stack, such as a stack contained in a piece of furniture. In some embodiments, the method and device further lift the stack vertically, whereby the next diaper in the stack is easily accessible to a user. In one embodiment, the furniture operates such that the diaper at the top of the stack is positioned in a way that reduces the amount of movement or bending a user must do to remove the diaper from the stack of diapers while changing a baby.

In some embodiments, the provided furniture device and methods operate in such a way that diapers may be stored in a stack without compressing the diapers against the top of the furniture. In one embodiment, the stack of diapers does not touch the lid of the furniture.

The present disclosure further provides a device and method for reloading or replenishing a stack of diapers contained within a piece of furniture.

In one embodiment of the diaper-dispensing piece of furniture, the furniture has an elongated interior space, a platform, a lead screw, a motor, a detector, and a processor. The elongated interior space has a cross-sectional area less than 100 square inches. The platform holds a stack of diapers. The screw may be a lead screw. Rotating the lead screw raises and lowers the platform within the piece of furniture, and thus moves the stack of diapers so they can be dispensed from the top of the piece of furniture. The motor rotates the lead screw. A detector may be positioned at the top of the interior space of the piece of furniture and is configured to detect when a diaper is dispensed from the interior space. A processor may be in communication with the detector and the motor, and the processor may be configured to activate the motor, thereby raising the platform, when the detector detects that a diaper has been dispensed from the piece of furniture.

In one embodiment, the motor may be a gear motor.

In one embodiment, the detector may be a photoelectric sensor, a reflective photoelectric sensor, photoelectric switch, proximity sensor, through-beam photoelectric sensor, laser sensor, or ultrasonic sensor.

In one embodiment, the top of the interior space can have a lid. In another embodiment, this lid may be operably coupled to a second detector configured to detect whether the lid is open or closed. This detector may be a snap action switch, action switch, magnetic switch, pressure sensor, rocker switch, slide switch, photoelectric sensor, photoelectric switch, proximity sensor, or reflective photoelectric sensor or other suitable sensor. In one embodiment, this detector may be configured to send a signal to a processor when the detector detects that the lid is open.

In one embodiment, the processor may be configured to activate the motor, thereby lowering the platform when the detector detects that the lid is open.

In one embodiment, a third detector may be configured to detect when the platform has been lowered to a point set as the low-level set point position within the interior space. This detector may be a snap action switch, action switch, magnetic switch, pressure sensor, rocker switch, slide switch, photoelectric sensor, photoelectric switch, proximity sensor, or reflective photoelectric sensor. In some embodiments, this detector may send a signal to the processor when it detects the platform has been lowered to the point set as the low-level set point position within the interior space.

In one embodiment, the processor may be configured to stop the motor, thereby stopping the platform, when the detector detects the platform has been lowered to the point set as the low-level set point position within the interior space.

In one embodiment, a sensor may be configured to determine the position of the platform. In some embodiments, this sensor may be a shaft encoder, laser sensor, ultrasonic sensor, or rotor encoder.

In one embodiment, the sensor configured to determine the position of the platform may be configured to transmit the position of the platform to a digital readout, such as a gauge or a wireless device, such as a smartphone, a tablet or a personal computer. In some embodiments, the sensor may be configured to transmit data encoding the position of the platform to a wireless device via the internet.

In one embodiment, the lead screw may be operably coupled to the platform using a nut.

In one embodiment, the lead screw may be operably mounted within the interior space of the piece of furniture using a flange bearing.

In one embodiment, the platform may be guided vertically within the interior space by a guide rod or a guide rail.

### BRIEF DESCRIPTION OF THE DRAWINGS

FIG. 1 is a photograph of one embodiment of the present disclosure.

FIG. 2 is schematic diagram showing elements of one embodiment of the present disclosure one embodiment of the present disclosure.

FIG. 3 is a photograph of one embodiment of the present disclosure.

### DETAILED DESCRIPTION

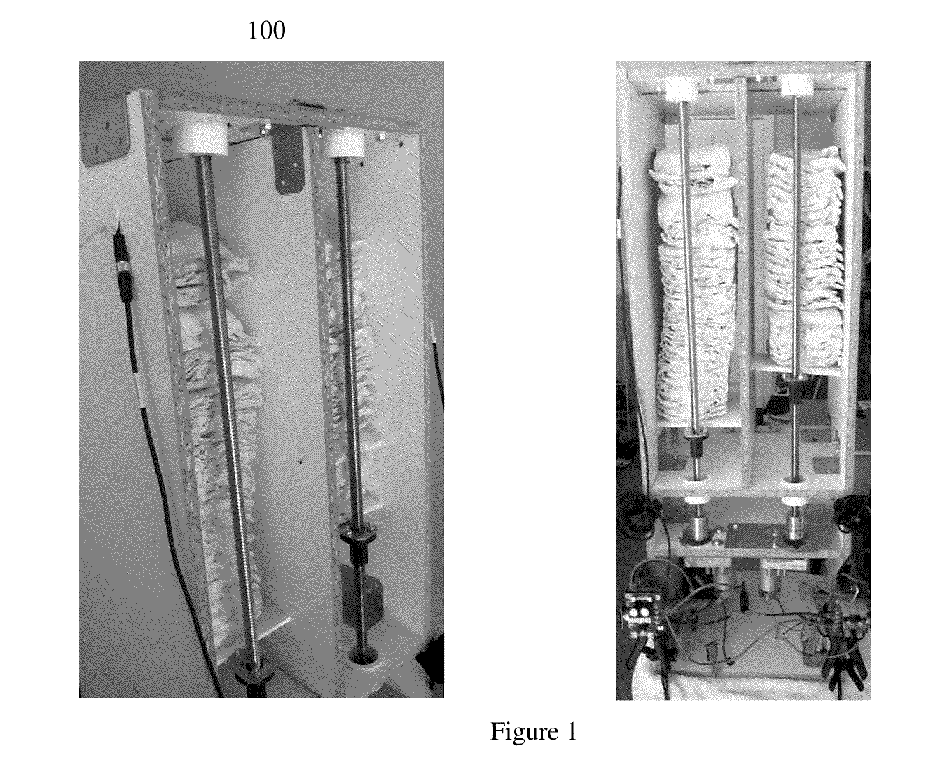

Disclosed herein are pieces of furniture for dispensing diapers and methods of making a piece of furniture for dispensing diapers. Changing a baby's diaper can be a difficult task. The baby might be squirming or crying. A person changing a baby's diaper wants to do so as quickly as possible. This disclosure allows a person changing a baby to have a diaper easily and conveniently accessible. The piece of furniture 100 depicted in FIG. 1 places a stack of diapers within reach. The piece of furniture may sense when a person has removed a diaper from the stack, and adjust the stack of diapers so another diaper is easily and conveniently accessible. The piece of furniture may also detect when doors and lids of the furniture are open or closed and modify its operation to allow a person to refill the stack of diapers. The diapers may be dispensed from the top of the piece of furniture, allowing a person to remove a diaper more easily than if the diaper to be dispensed was at the bottom of the diaper stack with the weight of the stack pressing down on it. The furniture may adjust the stack of diapers such that they are not compressed by the top of the furniture.

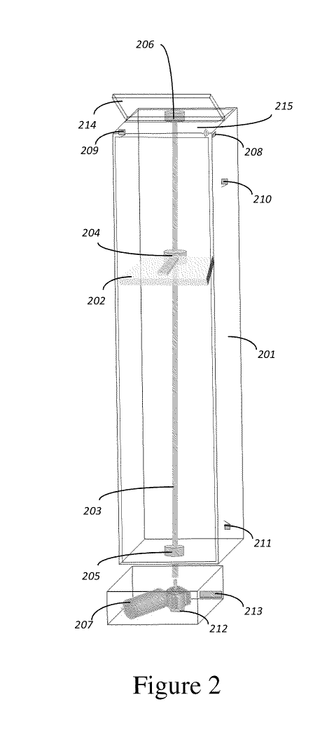

FIG. 2 is a schematic diagram showing elements of an embodiment. In one embodiment, diapers (not shown) are stacked on a moving platform 202 in an interior space 201 of the piece of furniture. The interior space 201 can be elongated and have a cross-sectional area of between 12.5 and 130 square inches. The cross sectional area is measured by a plane perpendicular to the elongated length and cutting through the interior space transversely. The movable platform 202 allows for the removal of a diaper from a top of the interior space 201. In some embodiments, removal of a diaper activates a detector 208, causing a motor 207 to adjust the platform 202 vertically. This adjustment advances another diaper from the stack into a readily accessible position for subsequent removal. The diapers can be stacked in any configuration, including vertically, horizontally or at any other angle. The diapers can also be stacked in a single orderly stack, multiple orderly stacks, or as single or multiple piles.

In some embodiments, the platform 202 can be coupled to a screw 203, such as a lead screw or a worm. A nut 204 can couple the platform 202 to the screw 203. In some embodiments, a motor 207 can rotate the screw, thereby adjusting the height of the platform in the interior space 201. The screw can be mounted to the interior of the furniture with bearings 205, 206.

In some embodiments, the furniture can include a processor 213. The processor can communicate with both the motor 207 and a detector 208 that is positioned at the top of the interior space. The detector 208 can sense when a diaper has been removed from the stack and send a signal to the processor 213. The processor 213 can then command the motor 207 to rotate in a manner that raises the platform 202. When the sensor 208 detects that the next diaper in the stack of diapers is appropriately positioned for dispensing, the sensor can send a signal to the processor 213. The processor 213 can then command the motor 207 to stop adjusting the height of the platform 202 above the bottom of the interior space.

The furniture can include an access aperture 215 in one or more panels creating the interior space. The aperture 215 can be for removing and stocking the interior space with diapers. In some embodiments, the appropriate height of the platform 202 can be such that a diaper can readily be removed from the access aperture 215.

In some embodiments, the platform 202 can be guided by guidance devices (not shown). Examples of guidance devices include guide rails, guide rods, and guide channels. A guidance device can be placed anywhere in or along the platform, including through a hole in the platform, along the sides of the platform or along corners of the platform. A guide channel can be a long vertical slit through a false wall in the back of the furniture. This guide channel can guide the platform 202 and cover the screw 203 and other moving parts so the stack of diapers does not interfere with the lifting mechanisms.

In some embodiments, the top panel 214 of the furniture can be a lid that is detachable or attached to the furniture with a hinge (not shown), allowing the stack of diapers to be replenished. Any of the side panels of the furniture can also be detachable or attached to the furniture with a hinge to form a door, allowing the stack of diapers to be replenished (not shown).

In some embodiments, a detector 209 can be placed near the removable panel or hinged door panel of the interior space. The detector 209 senses when the removable panel or hinged door panel 214 is removed or opened. This detector 209 may send a signal to the processor 213 indicating that this panel 214 has been removed or opened. The processor 213 may then activate the motor 207 to reverse the screw in a manner that lowers the platform 202. This allows for a user to replenish the supply of diapers. When the detector 209 senses that the removable panel or hinged door panel 214 is replaced or closed, the detector 209 may send a signal to the processor 213 indicating that the removable panel or hinged door panel 214 has been replaced or closed. The processor can then signal the motor 207 to turn the screw 203 and raise the platform 202 until the detector 208 senses that a diaper is at the appropriate height for dispensing.

The screw 203 can be any length between 10 and 100 inches and have a diameter between 0.1 and 4 inches. In some embodiments, the screw 203 is between 10 and 40 inches long. In other embodiments, the screw is between 10 and 20 inches long. The lead of the screw 203 can be between 0.05 and 2 inches. The lead, length, and diameter of the screw given here are exemplary and, in other embodiments, different leads, lengths, and diameters may be used. The screw 203 can have single or multiple starts. In some embodiments, the Thomson BSA SRA8-5008 lead screw can serve as the screw 203 in the piece of furniture.

The motor 207 turns the screw 203 at a rate that balances the convenience of speed with a quiet operation. The motor 207 is configured to raise the platform 202 quickly and quietly to move the next diaper in a stack of diapers into position for dispensing. The motor 207 is also configured to lower the platform 202 quickly and quietly so a user can refill the stack of diapers. In some embodiments, the motor 207 turns the screw 203 at a rate of 5-50 RPM while lifting the platform 202 and a rate of 5-100 RPM while lowering the platform 202. The screw 203 lifts the platform 202 at a speed of between 0.1-5 inches per second. The screw 203 lowers the platform 202 at a speed of 0.1-10 inches per second. The speed given here is exemplary and, in other embodiments, different speeds may be used. In some embodiments, the Rex Engineering Corporation's CXC motor can server as the motor 207 that turns the screw 203.

In some embodiments, a detector 211 can be placed near the bottom of the interior space. The detector 211 senses when the platform 202 has been lowered to a point set as the lower limit for the platform's 202 operable position. When the platform 202 reaches this point, it activates the detector 211, which sends a signal to the processor 213 to stop the motor from lowering the platform 202.

Similarly, in some embodiments, a detector 210 can be placed near the top of the interior space. The detector 210 senses when the platform 202 has been raised to a point set as the upper limit for the platform's 202 operable position. When the platform 202 reaches this point, it activates the detector 210, which sends a signal to the processor 213 to stop the motor from raising the platform 202.

In some embodiments, a detector 212 can be used to calculate the position of the platform 202 relative to the height of the screw 203. This detector can be a shaft encoder, an optical sensor, a photoelectric sensor, or any other type of detector capable of measuring distance or position. The detector 212 can communicate the position of the platform to the processor 213.

In some embodiments, the processor 213 can transmit the position of the platform 202 to a gauge or a digital display. Transmission of this data can occur wirelessly or via the internet. Such displays can allow a user to see the remaining diaper stock and alert a user when the diaper stack needs to be restocked. Devices capable of displaying such data include smart-phones, tablets and personal computers, for example.

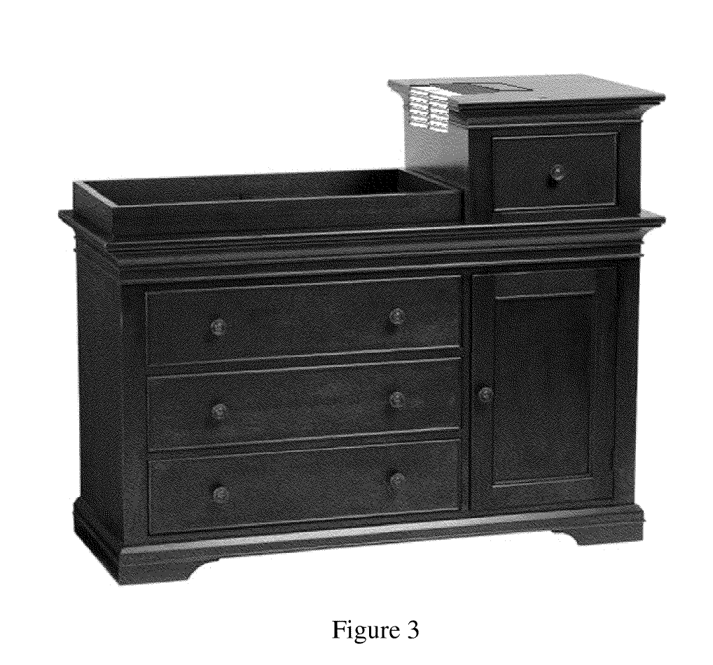

FIG. 3 is a photograph of one embodiment. The disclosed piece of furniture can be coupled to other pieces of furniture, such as a changing bed, a chest of drawers, or a cabinet. The furniture can be portable or mounted permanently. Multiple copies of the disclosed piece of furniture can be constructed together and contained within the same housing such that the combined piece of furniture can dispense multiple stacks of diapers, each stack having the same or different types of diapers. The multiple copies can function together or independently and have separate motors, screws, platforms, sensors, and processors.

In another embodiment of the disclosure, the platform 202 can be operably coupled to a motor (not shown), which operates a gear (a pinion) along a static linear gear bar (a rack). The motor turns the pinion such that the platform raises.

The disclosure also describes a method of dispensing diapers from a piece of furniture that has an elongated interior space, a platform, a lead screw, a motor, a detector, and a processor. A platform within the interior space holds stacked diapers. A person removes a diaper ready to be dispensed from the top of the interior space. A lead screw coupled to the platform to raises the platform within the interior space. A motor rotates the lead screw. A detector positioned at the top of the interior space detects when a user has removed a diaper from the interior space. The method further comprises a processor in communication with the detector and the motor activates the motor, thereby raising the platform, when the detector detects a diaper has been dispensed from the top of the interior space.

In another embodiment, a method of dispensing a diaper includes raising a stack of diapers within an elongated interior space of a piece of furniture, where the elongated space has a cross-sectional area less than 100 square inches. The raising of the stack of diapers includes rotating a lead screw attached to the platform. The method further comprises detecting when a diaper is dispensed form the interior space.

In another embodiment, the method of dispensing a diaper further comprises utilizing a gear motor that turns the lead screw, thereby raising and lowering the platform.

In another embodiment, the method of dispensing a diaper further comprises utilizing a sensor, such as a photoelectric sensor, a reflective photoelectric sensor, photoelectric switch, proximity sensor, through-beam photoelectric sensor, laser sensor, or ultrasonic sensor, detects when a diaper has been removed and sends a signal to the processor. The processor then activates the motor, thereby raising the platform so the next diaper is ready to be dispensed.

In another embodiment, the method of dispensing a diaper further comprises removing a diaper from the top of a piece of furniture, wherein the top of the interior space is a lid.

In another embodiment, the method of dispensing a diaper further comprises utilizing a second sensor, which detects whether the lid is open or closed.

In another embodiment, the method of dispensing a diaper further comprises utilizing a second sensor, which sends a signal to the processor when the lid is open. The processor then lowers the platform.

In another embodiment, the method of dispensing a diaper further comprises utilizing a second sensor, such as a snap action switch, action switch, magnetic switch, pressure sensor, rocker switch, slide switch, photoelectric sensor, photoelectric switch, proximity sensor, or reflective photoelectric sensor, which sends a signal to the processor when the lid is open. The processor then lowers the platform.

In another embodiment, the method of dispensing a diaper further comprises utilizing a third detector, which detects when the platform has been lowered to a point set as the low-level set point position within the interior space.

In another embodiment, the method of dispensing a diaper further comprises utilizing a third detector, which sends a signal to the processor when the third detector detects the platform has been lowered to the point set as the low-level set point position within the interior space. The processor then stops the motor, thereby stopping the platform.

In another embodiment, the method of dispensing a diaper further comprises utilizing a third detector, such as a snap action switch, action switch, magnetic switch, pressure sensor, rocker switch, slide switch, photoelectric sensor, photoelectric switch, proximity sensor, or reflective photoelectric sensor, which detects when the platform has been lowered to a point set as the low-level set point position within the interior space.

In another embodiment, the method of dispensing a diaper further comprises utilizing a position sensor, which determines the position of the platform. In some embodiments, the position sensor is selected from the group consisting of a shaft encoder, laser sensor, ultrasonic sensor, or rotor encoder.

In another embodiment, the method of dispensing a diaper further comprises the processor transmitting the position of the platform to a digital readout, such as a gauge, a display, or a wireless device selected from the group consisting of a smartphone, a tablet, or a personal computer. In some embodiments, the method of dispensing a diaper further comprises the processor transmitting the data encoding the position of platform to a wireless device over the internet.

In another embodiment, the method further comprises coupling the lead screw to the platform using a nut.

In another embodiment, the method further comprises mounting the lead screw to the interior space of the furniture with a flange bearing.

In another embodiment, the method further comprises guiding the platform within the interior space using a guide rod, guide rail, or guide channel.

As used herein, a detector may be understood to be any device that reads or responds to physical changes. Such changes can include physical contact, proximity to an object, position or rotation. Examples of sensors that can be used in the disclosure include a: snap-action switch, magnetic switch, pressure sensor, rocker switch, slide switch, photoelectric sensor, photoelectric switch, proximity sensor, reflective photoelectric sensor, through-beam photoelectric sensor, laser sensor, ultrasonic sensor, shaft encoder, or rotor encoder. Other detectors could also be used.

As used herein, a motor may be understood to be any type of motor capable of turning a screw, including a gear motor or a worm. Other motors and screws could also be used.

As used herein, a bearing may be understood to be a flange bearing, a ball bearing, a roller bearing, or a v-groove bearing system with centric spacers. Other bearings could also be used.

Although the present invention has been fully described in connection with embodiments thereof with reference to the accompanying drawings, it is to be noted that various changes and modifications will become apparent to those skilled in the art. Such changes and modifications are to be understood as being included within the scope of the present invention. The various embodiments of the invention should be understood that they have been presented by way of example only, and not by way of limitation. Although the invention is described above in terms of various exemplary embodiments and implementations, it should be understood that the various features and functionality described in one or more of the individual embodiments are not limited in their applicability to the particular embodiment with which they are described. They instead can, be applied, alone or in some combination, to one or more of the other embodiments of the invention, whether or not such embodiments are described, and whether or not such features are presented as being a part of a described embodiment. Thus the breadth and scope of the invention should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; and adjectives such as “conventional,” “traditional,” “normal,” “standard,” “known,” and terms of similar meaning, should not be construed as limiting the item described to a given time period, or to an item available as of a given time. But instead these terms should be read to encompass conventional, traditional, normal, or standard technologies that may be available, known now, or at any time in the future. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of the invention may be described or claimed in the singular, the plural is contemplated to be within the scope thereof unless limitation to the singular is explicitly stated. For example, “at least one” may refer to a single or plural and is not limited to either. The presence of broadening words and phrases such as “one or more,” “at least,” “but not limited to,” or other like phrases in some instances shall not be read to mean that the narrower case is intended or required in instances where such broadening phrases may be absent. The word “exemplary” is used herein to mean “serving as an example or illustration.” Any aspect or design described herein as “exemplary” is not necessarily to be construed as preferred or advantageous over other aspects or designs.

It will be appreciated that, for clarity purposes, the above description has described embodiments of the invention with reference to different functional units and processors. However, it will be apparent that any suitable distribution of functionality between different functional units, processing logic elements or domains may be used without detracting from the invention. For example, functionality illustrated to be performed by separate processing logic elements, or controllers, may be performed by the same processing logic element, or controller. Hence, references to specific functional units are only to be seen as references to suitable means for providing the described functionality, rather than indicative of a strict logical or physical structure or organization.

In the foregoing description of exemplary embodiments, reference is made to the accompanying drawings which form a part hereof, and in which it is shown by way of illustration specific embodiments in which the invention may be practiced. It is to be understood that other embodiments may be utilized and structural changes may be made without departing from the scope of the present disclosure.

It should be understood that the specific order or hierarchy of steps in the processes disclosed herein is an example of exemplary approaches. Based upon design preferences, it is understood that the specific order or hierarchy of steps in the processes may be rearranged while remaining within the scope of the present disclosure.

1. A piece of furniture for dispensing diapers comprising:
an elongated interior space having a cross-sectional area less than 100 square inches;
a platform within the interior space for holding stacked diapers, wherein the stacked diapers can be dispensed from a top of the interior space;
a lead screw coupled to the platform, wherein rotation of the lead screw raises the platform within the interior space;
a motor configured to rotate the lead screw;
a detector positioned at the top of the interior space and configured to detect when a diaper is dispensed from the interior space; and
a processor in communication with the detector and the motor, wherein the processor is configured to activate the motor, thereby raising the platform, when the detector detects a diaper has been dispensed from the top of the interior space.

2. The piece of furniture of claim 1, wherein the motor is a gear motor.

3. The piece of furniture of claim 1, wherein the detector is selected from the group consisting of a photoelectric sensor, a reflective photoelectric sensor, photoelectric switch, proximity sensor, through-beam photoelectric sensor, laser sensor, or ultrasonic sensor.

4. The piece of furniture of claim 1, further comprising a lid at the top of the interior space.

5. The piece of furniture of claim 4, wherein the lid is operably coupled to a second detector configured to detect whether the lid is open or closed.

6. The piece of furniture of claim 5, wherein the second detector is configured to send a signal to the processor and the processor is configured to activate the motor, thereby lowering the platform, when the second detector detects the lid has been opened.

7. The piece of furniture of claim 5, wherein the second detector is selected from the group consisting of a snap action switch, action switch, magnetic switch, pressure sensor, rocker switch, slide switch, photoelectric sensor, photoelectric switch, proximity sensor, or reflective photoelectric sensor.

8. The piece of furniture of claim 1, wherein a third detector has been configured to detect when the platform has been lowered to a point set as the low-level set point position within the interior space.

9. The piece of furniture of claim 8, wherein the third detector sends a signal to the processor, wherein the processor is configured to stop the motor, thereby stopping the platform, when the third detector detects the platform has been lowered to the point set as the low-level set point position within the interior space.

10. The piece of furniture of claim 10, wherein the third detector is selected from the group consisting of a snap action switch, action switch, magnetic switch, pressure sensor, rocker switch, slide switch, photoelectric sensor, photoelectric switch, proximity sensor, or reflective photoelectric sensor.

11. The piece of furniture of claim 1, further comprising a position sensor configured to determine the position of the platform and send a signal encoding the position of the platform to the processor.

12. The piece of furniture of claim 11, wherein the position sensor is selected from the group consisting of a shaft encoder, laser sensor, ultrasonic sensor, or rotor encoder.

13. The piece of furniture of claim 11, wherein the processor is configured to transmit the position of the platform to a digital readout, such as a gauge, a display, or a wireless device selected from the group consisting of a smartphone, a tablet, or a personal computer.

14. The piece of furniture of claim 11, wherein the processor is configured to transmit the data encoding the position of the platform to a wireless device via the internet.

15. The piece of furniture of claim 1, wherein the lead screw is operably coupled to the platform using a nut.

16. The piece of furniture of claim 1, wherein the lead screw is operably mounted within the interior space using a flange bearing.

17. The piece of furniture of claim 1, wherein the platform is guided vertically within the interior space by a guide rod, guide rail, or guide channel.
